(19) United States
(12) Patent Application Publication
(10) Pub. No.: US 2022/0062643 A1
(43) Pub. Date: Mar. 3, 2022

(54) Neuromodulatory Method for Treating Neurological Disorders

(71) Applicant: Dirk De Ridder, Dunedin (NZ)

(72) Inventor: Dirk De Ridder, Dunedin (NZ)

(21) Appl. No.: 17/209,149

(22) Filed: Mar. 22, 2021

Related U.S. Application Data
(60) Provisional application No. 62/992,854, filed on Mar. 20, 2020.

(51) Int. Cl. A61N 1/36; A61N 1/05

(52) U.S. Cl. CPC A61N 1/36171; A61N 1/36178; A61N 1/0551

(57) ABSTRACT

A method for the treatment of disorders of the nervous system using an external pulse generator or implantable pulse generator, in which one or more target nerves receive electrical stimulation signals that combine one or more frequency spectrums having a power spectral density per unit of bandwidth proportional to 1/f^{(-β)} noise, optionally combined with a regimen of applying such stimulation in a tonic manner or in a burst manner.

### RELATED APPLICATION

This application claims the benefit of U.S. Provisional Application No. 62/992,854 filed Mar. 20, 2020, which is incorporated herein by reference in its entirety.

### TECHNICAL FIELD OF THE INVENTION

The present invention relates to a method of using an EMD or IMD with a pulse generator, the method of which can be utilized to treat neurological conditions and/or disorders. More particularly, and not by way of limitation, the present invention is directed to a method for using 1/f^{(-β)} noise to treat neurological conditions and/or disorders.

### BACKGROUND OF THE INVENTION

Different firing modes or frequencies occur in the brain and/or other neuronal tissue, for example tonic firing and burst firing (irregular or regular burst firing). Such firing modes can be utilized for normal processing of information, however, alteration of the firing modes, may also lead to pathology.

For example, certain neurological conditions are associated with hyperactivity of the brain and can be traced to a rhythmic burst firing or high frequency tonic firing or hypersynchronous firing (e.g., tinnitus, pain, tremor, depression and epilepsy).

During the past decade, neuromodulation systems have been used to modulate various areas of the brain, spinal cord, or peripheral nerves (See, for example, U.S. Pat. Nos. 6,671,555; 6,690,974). These types of systems utilize tonic forms of electrical stimulation.

Recently the brain and its function has been conceived as a Bayesian prediction machine. This concept of brain functioning states that the brain predicts upcoming events based on prior beliefs (stored in memory) and updates these prior beliefs by sampling the environment via the senses. The prior belief that best fits the sensed environmental input survives and becomes the posterior belief, i.e. a percept, a thought, an idea, action or behavior. If there is no prediction error the posterior belief equals the prior belief and a status quo is maintained. If there is a prediction error the brain will attend to this prediction error. The predictions (prior beliefs) are generated in the beta frequency band and the prediction errors encoded in the gamma frequency band. Many pathologies are characterized by persisting prediction errors and or abnormal prior beliefs, i.e. by abnormal gamma and beta band activity respectively. These pathologies include, but are not limited pain, tinnitus, anxiety and depression, epilepsy, Parkinson disease, tremor, dystonia, addiction (food, alcohol, sex, gambling, illegal drugs), obsessive compulsive disorder, ADHD, Alzheimer and other dementias, personality disorders etc. The pathologies associated with abnormal predictions or persistent prediction errors include neurological, psychological and psychiatric disorders, as this is a general mechanism involved in those pathologies.

### BRIEF SUMMARY OF THE INVENTION

A method of stimulating nerve tissue of a patient using an EMD or IMD containing a pulse generator, the method comprising: storing, in the IMD containing a pulse generator, one first stimulation parameter that defines a frequency to be used as the lower bound of a frequency spectrum; storing, in the IMD containing a pulse generator, one second stimulation parameter that defines a frequency to be used as the upper bound of a frequency spectrum; generating, by IMD containing a pulse generator, a stimulus that comprises a frequency spectrum between the first stimulation parameter and second stimulation parameter, wherein the frequency and power of the frequency spectrum are proportional; providing the stimulus from the implantable IMD containing a pulse generator to at least one stimulation lead; and applying the stimulus to nerve tissue of the patient via at least one electrode of at least one stimulation lead wherein the stimulus is further defined as having a power spectral density per unit of bandwidth is proportional to 1/f^{(-β)}, wherein β is any other real number but excludes 0. A real number is a value of a continuous quantity that can represent a distance along a line. Real numbers include all the rational numbers, such as the integer −5 and the fraction 4/3, and all the irrational numbers, such as √2 (1.41421356 . . . , the square root of 2, an irrational algebraic number). Included within the irrationals are the transcendental numbers, such as it (3.14159265 . . . ). Further, wherein, the stimulus can be combined with at least one stimulation pulse to be repeated in a tonic manner, the stimulus can be combined with a burst stimulus that comprises a plurality of groups of spike pulses or the stimulus is modulated at any specific frequency either by selective power increase, envelope modulation or adding more tonic or burst stimuli of this frequency. A stimulation design is developed that specifically modulates those beta related predictions and gamma related prediction errors, called 1/f^{(-β)} stimulation.

A method of stimulating nerve tissue of a patient using an IMD containing a pulse generator, the method comprising: storing, in the IMD containing a pulse generator, one first stimulation parameter that defines a frequency to be used as the lower bound of a frequency spectrum; storing, in the IMD containing a pulse generator, one second stimulation parameter that defines a frequency to be used as the upper bound of a frequency spectrum; generating, by IMD containing a pulse generator, a stimulus that comprises a frequency spectrum between the first stimulation parameter and second stimulation parameter, wherein the frequency and power of the frequency spectrum are proportional; providing the stimulus from the implantable IMD containing a pulse generator to at least one stimulation lead; and applying the stimulus to nerve tissue of the patient via at least one electrode of at least one stimulation lead wherein the stimulus is further defined as having a power spectral density per unit of bandwidth is proportional to 1/f^{(-β)}, wherein β is any real number but excludes 0. Further, where the stimulus is combined with at least one stimulation to be repeated in tonic manner or burst stimulus that comprises a plurality of groups of spike pulses, either individually charge balanced, or charge balanced at the end of the group of monophasic spikes.

A method of stimulating nerve tissue of a patient using an IMD containing a pulse generator, the method comprising: storing, in the IMD containing a pulse generator, one first stimulation parameter that defines a frequency to be used as the lower bound of a frequency spectrum; storing, in the IMD containing a pulse generator, one second stimulation parameter that defines a frequency to be used as the upper bound of a frequency spectrum; storing, in IMD containing a pulse generator, one third stimulation parameter that defines a frequency at which a peak of a pre-determined amplitude is to occur; generating, by IMD containing a pulse generator, a stimulus that comprises a frequency spectrum between the first stimulation parameter and second stimulation parameter, wherein the power spectral density per unit of bandwidth is proportional to 1/f^{(-β)}, wherein β is excludes 0, and wherein a peak of a predetermined amplitude occurs at the frequency defined by the third stimulation parameter; providing the stimulus from the IMD containing a pulse generator to at least one stimulation lead; and applying the stimulus to nerve tissue of the patient via one or several electrodes of the at least one stimulation lead. Further, the noise stimulus can be combined with at least one stimulation pulse to be repeated in a tonic manner and with a burst stimulation that comprises a plurality of groups of spike pulses, either individually charge balanced, or charge balanced at the end of the group of monophasic spikes. Further, the peak can occur at a frequency between 0 and 30 Hertz.

### BRIEF DESCRIPTION OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In the figures, elements having the same designation have the same or similar functions.

FIGS. 1A through 1D illustrate 1/f^{(-β)} noise. FIG. 1A shows an exemplary 1/f^{(-β)} noise spectrum generated by a power source were β has a value of 1. FIG. 1B shows an exemplary 1/f^{(-β)} noise spectrum where β has a value of 1. FIG. 1C shows 1/f^{(-β)} noise spectrum generated by a power source were β has a value of 2. FIG. 1D shows an exemplary 1/f^{(-β)} noise spectrum where β has a value of 2.

FIGS. 2A and 2B illustrate 1/f^{(-β)} noise where β has a value of zero.

FIGS. 3A-3J illustrate example electrical stimulation leads that may be used to electrically stimulate neuronal tissue.

FIG. 4 is an example of a system of electrical stimulation leads that may be used to electrically stimulate neuronal tissue.

FIG. 5 depicts a stimulation system that can measure or detect given neuronal signals that can be used to modulate the 1/f^{(-β)} noise stimulation according to one representative embodiment.

FIG. 6 depicts a stimulation system that can sense and/or monitor sleep stage that can be used to alter therapy.

FIG. 7 depicts modules within the memory of FIG. 6.

### DETAILED DESCRIPTION

The method and system described herein relate to stimulating nerve tissue to treat a neurological disease and/or condition. Using an IMD containing a pulse generator, a stimulus is created that comprises a structured noisy signal.

The brain functions are structured following a 1/f^{β}. The spectral exponent β is typically close to 1 when brain function is measured in the awake state with electroencephalography (EEG), magnetoencephalography (MEG) or functional magnetic resonance imaging (fMRI). However, when recording directly from the brain with local field potentials or electrocorticography (ECoG) the β is typically closer to 2. When the level of consciousness decreases, as in sleep, anesthesia or coma the β exponent also increases.

The method and system described herein comprises a 1/f^{(-β)} noise stimulation. The 1/f^{(-β)} noise specifically targets pathological beta and gamma activity reflective of predictions and prediction errors. Thus, the 1/f^{(-β)} noise stimulation specifically targets and disrupts pathological beta and gamma activity associated with disorders of the nervous system.

The method and system described herein relate to stimulating nerve tissue to treat a neurological disease and/or condition. Using an EMD or IMD containing a pulse generator, a stimulus is created that produces a signal with a frequency spectrum having a power spectral density per unit of bandwidth proportional 1/f^{(-β)}, wherein β any real number but excludes 0.

β can be, for example, any real, natural, integer, rational, irrational, complex or fluctuating number. For example, β=1 or β=2, but as stated, it can also be irrational numbers such as π, ε, or φ. The stimulus is provided from the EMD or IMD containing a pulse generator to at least one stimulation lead; and applied to nerve tissue of the patient via one or several electrodes of the at least one stimulation lead. The frequency range is between 0.01 Hz and 1000 Hz, but typically will be within 0.1 Hz and 100 Hz. The slope, determined by the β exponent, can plateau at any frequency but typically between 40 and 100 Hz

Yet further, the stimulus can be combined with at least one pulse stimulus repeated in a tonic manner or a burst stimulus that comprises a plurality of groups of spike pulses.

Still further, the stimulus can be modulated at any specific frequency, either by selective power increase, envelope modulation or adding more tonic or burst stimuli of this frequency.

A first stimulation parameter that defines a frequency having a lower bound of a frequency spectrum (0.01 Hz) and a second stimulation that defines a frequency having an upper bound of a frequency spectrum (1000 Hz) can be stored in a controller or EMD or IMD containing a pulse generator and such controller can be used to generate a stimulus that comprises a frequency spectrum between the first and second stimulation parameters such that the frequency and power of the frequency spectrum are proportional.

The higher part of the stimulation can level off with a plateau, so that the amplitude in the high frequencies does not become excessive. This means that the β of the high frequencies, i.e. the 1/f^{(-β)} approaches but does not equal 0. The leveling off at high frequencies can typically be at 40-100 Hz.

Currently, stimulation generated by EMD or IMDs are not physiological, thus not similar to the endogenous electrical signals generated by the brain, which are noisy. The exogenous rhythmic electrical signals generated by the EMD or IMDs may result in epileptic events and/or brain habituation to stimulation signals over time. The inventor is the first to describe a stimulation design composed of parameters in which a 1/f^{(-β)} (one over frequency raised to the power of minus β) noise is used to disrupt abnormal brain wave frequency band activity to treat a neurological condition. The noisy structure of the stimulation prevents epilepsy and habituation.

The following section more generally describes an example of a procedure for treatment using a 1/f^{(-β)} noise known herein as “One-over-f-raised-to-the-power-of-minus-beta” noise. In addition to other advantages, 1/f^{(-β)} noise allows for the optimization of many parameters that provide certain effects including (without limitation) the following: a set and/or range of stimulation waveforms and/or protocols that can completely eliminate neurological disease/disorder; a set and/or range of efficacious stimulation waveforms and/or protocols that requires the lowest voltage; and a waveform and/or protocol that maintains treatment efficacy over long periods of time such as a waveform and/or protocol that can prevent habituation or adaptation, and a waveform and/or protocol that is anti-epileptic.

Still further, the generated 1/f^{(-β)} noise signal can be, for example, filtered, combined, or otherwise processed whereby the generated 1/f^{(-β)} noise is utilized as a background noise signal over another signal with a spectral peak at a selected frequency. For example, the spectral peak can be an alpha peak, delta peak, beta peak, gamma peak and/or theta peak. Any one or more of these peaks can be added to the 1/f^{(-β)} noise. The peaks can be generated using typical known frequencies or the peaks can be individualized for each patient.

Yet further, the 1/f^{(-β)} noise can be combined with standard tonic and/or burst stimulation to further enhance the optimization or prevent habituation. Combinations of tonic and/or burst stimulation are known in the art, for example, U.S. Pat. Nos. 7,734,340 and 8,364,273, which are incorporated by reference in their entirety.

The predetermined site for stimulation can include, for example, peripheral neuronal tissue and/or central neuronal tissue. Peripheral neuronal tissue can include an autonomic (sympathetic and parasympathetic) or somatic peripheral nerve, a nerve root or root ganglion, or any peripheral neuronal tissue associated with a given dermatome or nerve field, or any neuronal tissue that lies outside the central neuronal tissue such as the brain, brainstem or spinal cord.

1. 1/f^{(-β)} (also known herein as “One-over-f-raised-to-power-of-minus-beta”)

A noise signal can be described as a signal that is generated according to a random process. Purely random processes include only 1/f^{β} with β=0. In practice, various algorithms (e.g., in software executed on a processor) are employed to simulate a given random process to generate a “pseudo-random” signal, i.e. structured noise, where the generated pseudo-random signal possesses similar characteristics with signals corresponding and correlated to a random process. The characteristics of a particular noise signal depend upon the underlying process generating the noise signal. For example, the power spectral density or power distribution in the frequency domain may be employed to characterize the random process and, hence, also characterize a corresponding time-domain noise signal. The classification of the power spectral density of a noise signal may be described in reference to the noise signal's algorithm associated with different types of power spectral densities.

According to these conventions, the power spectral density can be defined herein as 1/f^{(-β)} noise. For this equation, f represents frequency and β is a value selected to characterize the noise signal.

1/f^{(-β)} noise has advantages over current stimulation paradigms, because of the ability of 1/f^{(-β)} noise to disrupt pathological high frequency activity, such as beta and gamma activity. These advantages arise due to the unique features of 1/f^{(-β)}. These advantages include permitting low amplitudes to be used, permitting stability, permitting adaptive flexibility via stochastic resonance, drowning out rhythmic pathological signals in beta and gamma, and permitting better analogue to digital conversion at receptor site. 1/f^{(-β)} noise stabilizes naturally occurring background activity and removes pathological activity as well, while improving analogue to digital conversion.

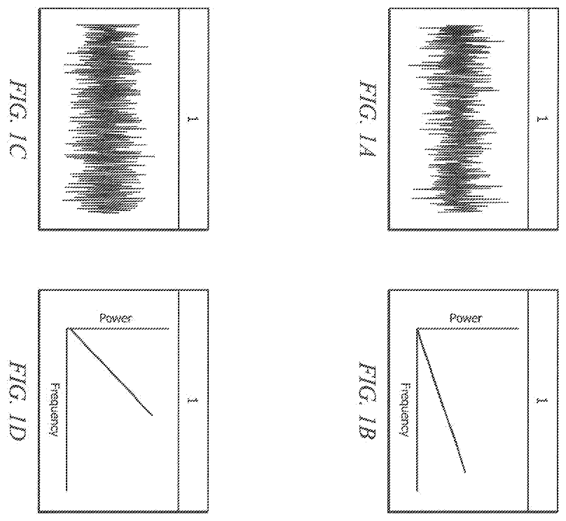

FIG. 1 shows the 1/f^{(-β)} noise relationships as β changes. FIGS. 1A and 1B show the 1/f^{(-β)} noise when β=1, while FIG. 1C and FIG. 1D show 1/f^{(-β)} noise when β=2. As can be seen in comparing FIGS. 1B and 1D, as β increases, the slope of 1/f^{(-β)} noise increases.

The value of β can be, for example, any real, natural, integer, rational, irrational or complex number. For example, the spectral density for white noise is flat (β=0). For 1/f^{(-β)} noise, β can equal 1, 2 or it can be greater than 2 (eg 3, 4, 5 . . . etc). Suitable non-integer β values about 1 include 0.5, 0.6, 0.7, 0.8, 0.9, 1.0, 1.1, 1.2, 1.3, 1.5, or any values there between for some embodiments. Likewise, suitable non-integer β values about 2 can include 1.5, 1.6, 1.7, 1.8, 1.9, 2.0, 2.1, 2.2, 2.3, 2.4, 2.5 or any value there between for some embodiments. In general, for non-integer values about an integer X, β values can include Y.5, Y.6, Y.7, Y.8, Y.9, X, X.1, X.2, X.3, X.4, X.5, where Y equals X minus 1.

Abnormal electrical and/or neural activity is associated with different diseases and disorders in the central and peripheral nervous systems. In addition to a drug regimen or surgical intervention, potential treatments for such diseases and disorders include using a stimulating system that includes an external medical device (EMD) or implantable medical device (IMD) for electrical stimulation of a portion or all of the patient's body tissue. For example, an EMD may be an external pulse generator EPG (such as an external trial stimulator or an external waveform generator. An EMD may also be an external device that provides transcranial electric noise stimulation.

For example, an IMD can be an implantable pulse generator (IPG) or a radio frequency pulse generator (RF), either of which can provide electrical stimulation to nervous tissue. Those skilled in the art know that both IPG and RF systems contain internal pulse generators for stimulating nervous tissue. One difference between an IPG and a RF system is the location of each system's power source. An IPG's power source is usually a battery and is contained within the IPG, which is implanted in the patient's body. A RF system's power source is external to the human body, such that power is transmitted using radio frequency waves that are inductively linked to a radio frequency antennae or receiver coupled to or in the RF device, which is located in the human body. An example IPG system is the Algovita system formerly marketed by Nuvectra®. An example RF system is Nalu®'s neurostimulation system. In particular, an IMD may electrically stimulate a target neuronal tissue location by the selective application of controlled electrical input signals to one or more electrodes coupled to or placed in proximity to the patient's neuronal tissue. Such electrical input signals may be applied to the patient's neuronal tissue in order to treat a neurological disease, condition, or disorder.

The response of nonlinear systems to signal (normal or abnormal) may be optimized or treated by combining the signal with a non-negligible level of noise. This optimization or increased sensitivity cause by a noisy (stochastic) signal is referred to herein as “Stochastic Resonance.” Said another way, when a noisy (stochastic) signal is intentionally injected or added into a nonlinear (for example, neurological) system, the system's sensitivity increases. This optimization or increased sensitivity is also called “dithering.” Dithering is a form of stochastic resonance.

A specific example of dithering is 1/f^{(-β)} noise. By adding a 1/f^{(-β)} a nervous system's ability to process or treat an abnormal or weak signal created by the nervous system is enhance. In this dithering example, the nervous system becomes more sensitive or optimized due to the 1/f^{(-β)} noise. This optimization or increased sensitivity allows the nervous system to process the abnormal or weak signal in a way that today's neuromodulation signals do not. Dithering improves the brain's ability to correctly process weak or abnormal signals corresponding to certain diseases states from a normal signal. Non-limiting examples of disease states that have weak (weak or abnormal), such as those signals resulting from partial or complete deafferentation or abnormal signals are deprivation of stimulus input. This is manifest in disorders such as chronic pain, tinnitus, Parkinson disease, depression, anxiety, paresthesia, addiction, OCD and others. 1/f^{(-β)} noise allows the brain over time to correctly predict the weak and/or abnormal signals as signals to process in a predictable manner. Those skilled in the art understand that the previous examples of disease states are not meant to be limiting examples and that treatment using a 1/f^{(-β)} noise signal will apply to disease states that contains weak or abnormal signals.

1/f^{(-β)} noise is also seen in “Composite Noise Stimulation (CNS). CNS, which is described in more detail in the inventor's co-pending application XXXXXXX, which is incorporated by reference herein in full, is a signal that combines two kinds of structured noise, including 1/f^{β} noise and 1/f^{(-β)} noise. Those skilled in the art will recognize that the choice of 1/f^{(-β)} stimulation or CNS depends on the goals of the treatment paradigm, the patient and the outcome desired, and does not change the invention herein.

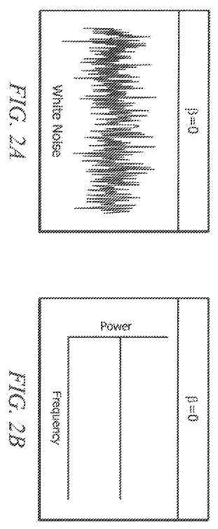

For a system to exhibit stochastic resonance there needs to be a threshold that must be exceeded in order to activate the system. When the input signal is not strong enough to exceed a threshold, noise, even if in small amounts, added either to the system or the signal may occasionally suffice to trigger activation. Typically, this type of phenomenon is associated with 1/f^{(-β)} noise, where β equals zero, which is sometimes referred to as “pure noise” or “white noise” as shown in FIGS. 2A and 2B. Adding 1/f^{(-β)} noise signals improves signal quality and mitigates artefact.

Those skilled in the art know that in current stimulation paradigms, stimulation signals are typically repetitive square wave pulses. Over time, such a repetitive electrical stimulation signal is dissimilar to the brain's own naturally occurring signals. Such repetitive signals may become less effective as the brain “filters out,” “ignores”, habituates or accommodates to the repetitive signal. It is also known as tolerance (to stimulation). Hence, a problem with standard electrical stimulation parameters used today is habituation, because the electrical stimulation parameters result in a very predictive repetitive electrical signal and thus, the brain habituates to the signal or adapts.

One way to have an electrical stimulation signal to resemble the brain's own signals is to utilize a stimulation paradigm that provides a signal that is correlated to that of the brain's normal signals. It is believed that there exists at least some naturally occurring signals within the nervous system that closely resemble 1/f^{β}. The disadvantage of 1/f^{β} stimulation is that beta and gamma frequencies have low power, inherent to the fact that the power decreases inversely with frequency. Because of this, the efficacy of 1/f^{(-β)} noise signals applied to neuronal tissue is improved, specifically in the higher frequencies such as beta and gamma, because in 1/f^{(-β)} stimulation the power is proportional to the frequency.

To further the effectiveness of the given therapy, one can modulate the 1/f^{(-β)} noise paradigm by adding specific peak frequencies to the 1/f^{(-β)} noise signal that are known or associated with given brain areas. For example, one can add an alpha frequency peak to the 1/f^{(-β}) noise to stimulate primary and secondary cortical areas; add a theta frequency peak to the 1/f^{(-β}) noise to stimulate the cingulate, hippocampus, amygdala; add a delta frequency peak to the 1/f^{(-β)} noise to stimulate the brainstem, ventral tegmental area (VTA), nucleus accumbens/ventral medial prefrontal cortex (VMPFC).

These additional peak frequencies that are added to the 1/f^{(-β)} noise can be obtained from the individual by EEG or MEG measurements or any other measurement to obtain the individual peak frequency or the frequencies can be obtained from a database, for example a database containing a list of given frequencies and spectral structures for a brain structure or brain area. The frequency for each brain area, for example, each Brodmann area, can be easily calculated by defining a Brodmann area in source space and performing a spectral analysis for that area using any software (i.e., sLORETA) to perform source analysis.

Still further, the 1/f^{(-β)} noise can be modified by using multiple poles or electrodes. For example, the stimulation paradigm is either sequentially cycled or randomly cycles through the specific or alternating poles or electrodes upon the stimulation lead.

The 1/f^{(-β)} noise can also be selected to specifically activate or inactivate a brain area or brain network. It can be chosen so as to not be normalizing, but instead to be non-physiological to compensate for overactivity or hypoactivity. This can be combined with normal, inversely proportional physiological 1/f^{β} noise parameters, as mentioned in the co-pending XXX application.

II. Patient Selection

Subjects to be treated according to some representative embodiments can be selected, identified and/or diagnosed based upon the accumulation of physical, chemical, and historical behavioral data on each patient. One of skill in the art is able to perform the appropriate examinations to accumulate such data. One type of examination can include neurological examinations, which can include mental status evaluations, which can further include a psychiatric assessment. Other types of assessments for movement disorders may include such assessments for example using the Unified Parkinson's Disease Rating Scale (UPDRS). Still further, other types of examinations can include, but are not limited to, motor examination, cranial nerve examination, cognitive assessment and neuropsychological tests (i.e., Minnesota Multiphasic Personality Inventory, Beck Depression Inventory, or Hamilton Rating Scale for Depression). Other types of assessment for tinnitus, for example, can include but are not limited to Numeric Rating Scales (NRS) or Visual Analogue Scales (VAS) and Tinnitus Handicap Inventory (THI), Tinnitus Questionnaire, Tinnitus Functional Index, for pain VAS or NRS, as well as Pain Vigilance and Awareness Questionnaire, Pain Catastrophizing Scale, McGill Pain questionnaire and others, for depression Beck Depression Inventory or Hospital Anxiety and Depression Scale. In addition to neurological testing, routine hematological and/or biochemistry testing may also beperformed.

In addition to the above examinations, imaging techniques can be used to determine normal and abnormal brain function that can result in disorders. Thus, once the patient is identified from the above clinical examinations, imaging techniques can be further utilized to provide the region of interest in which the electrodes are to be implanted. Functional brain imaging allows for localization of specific normal and abnormal functioning of the nervous system. This includes electrical methods such as electroencephalography (EEG), magnetoencephalography (MEG), single photon emission computed tomography (SPECT), as well as metabolic and blood flow studies such as functional magnetic resonance imaging (fMRI), and positron emission tomography (PET) which can be utilized to localize brain function and dysfunction.

III. Implantation of Stimulation Leads

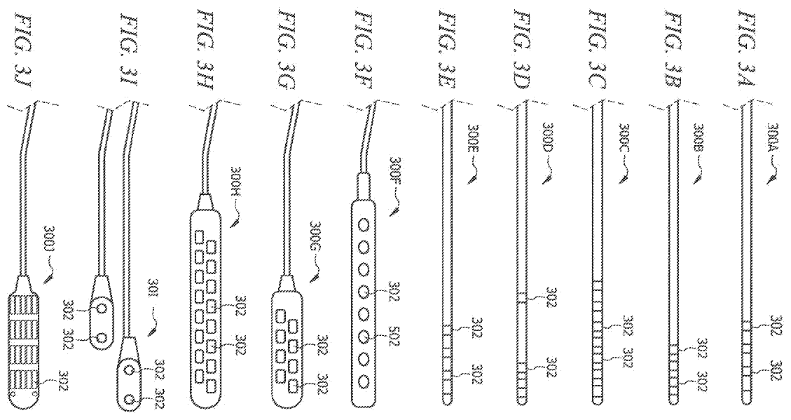

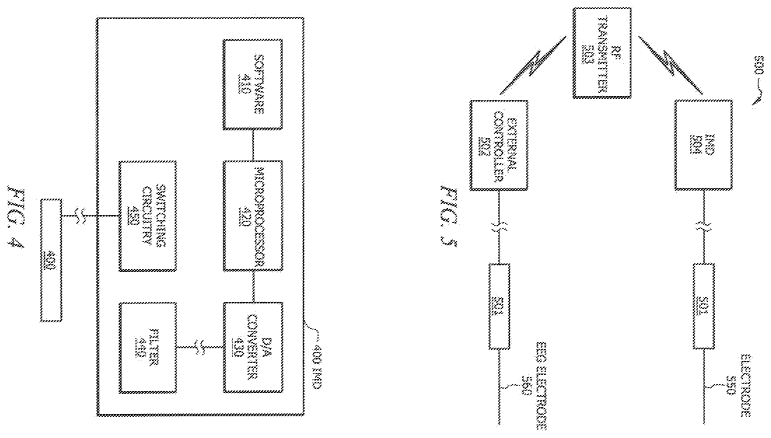

One or more stimulation leads 100, as shown in FIGS. 3A-3J are implanted such that one or more stimulation electrodes 302 of each stimulation lead 300 are positioned or disposed near, adjacent to, directly on or onto, proximate to, directly in or into or within the target tissue or predetermined site. The leads shown in FIG. 3 are exemplary of many commercially available leads, such as deep brain leads, percutaneous leads, paddle leads, etc. Examples of commercially available stimulation leads includes a Nuvectra® percutaneous lead or various laminotomy or paddle leads, such as Nuvectra's laminotomy lead, that are currently on market with which those skilled in the art are familiar. For the purposes described herein and as those skilled in the art will recognize, when an embedded stimulation system, such as the Bion®, is used, it is positioned similar to positioning the lead 100.

Techniques for implanting stimulation electrodes are well known by those of skill in the art and may be positioned in various body tissues and in contact with various tissue layers; for example, deep brain, cortical, subdural, subarachnoid, epidural, cutaneous, transcutaneous and subcutaneous implantation is employed in some embodiments.

A. Brain

Central neuronal tissue includes brain tissue, spinal tissue or brainstem tissue. Brain tissue can include the frontal lobe, the occipital lobe, the parietal lobe, the temporal lobe, the cerebellum, or the brain stem. More specifically, brain tissue can include subcortical targets, for example, thalamus/sub-thalamus (i.e., thalamic nuclei, medial and lateral geniculate body, intralaminar nuclei, nucleus reticularis, pulvinar, subthalamic nuclei (STN), habenula etc) basal ganglia (i.e., putamen, caudate nucleus, globus pallidus), hippocampus, amygdala, hypothalamus, epithalamus, mammilary bodies, ventral tegmental area (VTA), nucleus accumbens, substantia nigra, corpus callosum, fornix, internal capsula, anterior and posterior commissural, cerebral peduncles etc. Brain tissue also includes cerebellum, cerebellar peduncles, and cerebeller nuclei such as fastigial nucleus, globose nucleus, dentate nucleus, emboliform nucleus. Further, in addition to grey matter, also white matter tracts may be targeted, such as the anterior limb of the interior capsula, the medial forebrain bundle, the cingulum bundle and other white matter tracts connecting different parts of the brain. Still further, in addition to the above mentioned subcortical targets, brain tissue also includes cortical targets, for example, auditory cortex, prefrontal cortex, the dorsolateral prefrontal cortex, the ventromedial prefrontal cortex, the cingulate cortex, subcallosal area, anterior cingulate cortex, the subgenual anterior cingulate cortex, the motor cortex and the somatosensory cortex. The somatosensory cortex comprises the primary, the secondary somatosensory cortex, and the somatosensory association complex. Still further, the somatosensory cortex also includes Brodmann areas 1, 2, 3, 5, and 7. Yet further, brain tissue can include various Brodmann areas for example, but not limited to Brodmann area 9, Brodmann area 10, Brodmann area 24, Brodmann area 25, Brodmann area 32, Brodmann area 39, Brodmann area 41, Brodmann area 42, and Brodmann area 46.

While not being bound by the description of a particular procedure, patients who are to have an electrical stimulation lead or electrode implanted into the brain for deep brain stimulation, generally, first have a stereotactic head frame, such as the Leksell, CRW, or Compass, mounted to the patient's skull by fixed screws. Subsequent to the mounting of the frame, the patient typically undergoes a series of magnetic resonance imaging sessions, during which a series of two-dimensional slice images of the patient's brain are built up into a quasi-three-dimensional map in virtual space. This map is then correlated to the three-dimensional stereotactic frame of reference in the real surgical field. In order to align these two coordinate frames, both the instruments and the patient must be situated in correspondence to the virtual map. The current way to do this is to rigidly mount the head frame to the surgical table. Subsequently, a series of reference points are established to relative aspects of the frame and patient's skull, so that either a person or a computer software system can adjust and calculate the correlation between the real world of the patient's head and the virtual space model of the patient MRI scans. The surgeon is able to target any region within the stereotactic space of the brain with precision (e.g., within 0.5 mm). Initial anatomical target localization is achieved either directly using the MRI images or functional imaging (PET or SPECT scan, fMRI, MSI), or indirectly using interactive anatomical atlas programs that map the atlas image onto the stereotactic image of the brain. As is described in greater detail elsewhere in this application, the anatomical targets or predetermined site may be stimulated directly or affected through stimulation in another region of the brain, which is functionally or structurally connected to the target area.

In addition to deep brain stimulation, cortical stimulation can also be used to stimulate various brain tissues. Any of the stimulation leads illustrated in FIGS. 3A-3J can be used for cortical stimulation, as well as any other cortical electrode or electrode array. For implanting conventional cortical electrodes, it typically requires a craniotomy under general anesthesia to remove a relatively large (e.g., thumbnail-sized or larger) window in the skull. A pilot hole (e.g., 4 mm or smaller) can be formed through at least part of the thickness of the patient's skull adjacent a selected or predetermined site. In certain embodiments, the pilot hole can be used as a monitoring site.

The location of the pilot hole (and, ultimately the electrode received therein) can be selected in a variety of fashions, for example, the physician may use anatomical landmarks, e.g., cranial landmarks such as the bregma or the sagittal suture, to guide placement and orientation of the pilot hole or the physician may use a surgical navigation system. Navigation systems may employ real-time imaging and/or proximity detection to guide a physician in placing the pilot hole and in placing the electrode in the pilot hole. In some systems, fiducials are positioned on the patient's scalp or skull prior to imaging and those fiducials are used as reference points in subsequent implantation. In other systems, real-time MRI or the like may be employed instead of or in conjunction with such fiducials. A number of suitable navigation systems are commercially available, such as the STEALTHSTATION TREON TGS sold by Medtronic Surgical Navigation Technologies of Louisville, Colo., U.S.

Once the pilot hole is formed, the threaded stimulation lead may be advanced along the pilot hole until the contact surface electrically contacts a desired portion of the patient's brain. If the stimulation lead is intended to be positioned epidurally, this may comprise relatively atraumatically contacting the dura mater; if the electrode is to contact a site on the cerebral cortex, the electrode will be advanced to extend through the dura mater. Thus, the lead may be placed epidurally or subdurally for cortical stimulation.

B. Spinal Cord and/or Peripheral Nerves

Peripheral nerves can include, but are not limited to cranial nerves, somatic (spinal) nerves and autonomic nerves. Cranial nerves include the olfactory nerve, optic nerve, oculomotor nerve, trochlear nerve, trigeminal nerve, abducens nerve, facial nerve, vestibulocochlear (auditory) nerve, glossopharyngeal nerve, vagal nerve, accessory nerve, and hypoglossal nerve. Peripheral spinal nerves can be any nerve from C2 to S5, i.e. cervical, thoracic, lumbar and sacral nerves, and include both afferent and efferent components. Some spinal nerve branches have names such as occipital nerve (e.g., suboccipital nerve, the greater occipital nerve, the lesser occipital nerve), the greater auricular nerve, the lesser auricular nerve, brachial plexus, radial axillary nerves, musculocutaneous nerves, radial nerves, ulnar nerves, median nerves, intercostal nerves, lumbosacral plexus, sciatic nerves, common peroneal nerve, tibial nerves, sural nerves, femoral nerves, gluteal nerves, thoracic spinal nerves, obturator nerves, digital nerves, pudendal nerves, plantar nerves, saphenous nerves, ilioinguinal nerves, genitofemoral nerves, and iliohypogastric nerves. Furthermore, peripheral neuronal tissue can include but is not limited to peripheral nervous tissue associated with a dermatome. In other embodiments the peripheral nerve can be an autonomic nerve, either from the sympathetic or parasympathetic system. Some of the autonomic nerves run in named nerves such as the vagus (=vagal) nerve, the different splanchnic nerves, celiac nerve, mesenteric plexus, phrenic nerve. In other embodiments the stimulation can be applied to the dorsal root ganglion of any one or more cervical (C1, C2, C3, C4, C5, C6, C7 and C8), thoracic (T1, T2, T3, T4, T5, T6, T7, T8, T9, T10, T11, T12), lumbar (L1, L2, L3, L4. L5) or sacral (S1, S2, S3, S4, S5) nerves

Spinal tissue can include the ascending and descending tracts of the spinal cord, more specifically, the ascending tracts that comprise intralaminar neurons or the dorsal column. For example, the spinal tissue can include neuronal tissue associated with any of the cervical vertebral segments (C1, C2, C3, C4, C5, C6, C7 and C8) and/or any tissue associated with any of the thoracic vertebral segments (T1, T2, T3, T4, T5, T6, T7, T8, T9, T10, T11, T12) and/or any tissue associated with any of the lumbar vertebral segments (L1, L2, L3, L4. L5) and/or any tissue associated with the sacral vertebral segments (S1, S2, S3, S4, S5). More specifically, the spinal tissue is the dorsal column or the dorsal root ganglia of the spinal cord. The brainstem tissue can include the medulla oblongata, pons or mesencephalon, more particular the posterior pons or posterior mesencephalon, Lushka's foramen, and ventrolateral part of the medulla oblongata.

In other embodiments, the stimulation leads are positioned in communication with the neuronal tissue of the spinal cord, more specifically, the dorsal root ganglia or the dorsal column of the spinal cord. For example, stimulation electrodes are commonly positioned external to the dura layer surrounding the spinal cord. Stimulation on the surface of the cord is also contemplated, for example, stimulation may be applied to the spinal cord tissue as well as to the nerve root entry zone. Stimulation electrodes may be positioned in various body tissues and in contact with various tissue layers; for example, subdural, subarachnoid, epidural, and cutaneous, and/or subcutaneous implantation is employed in some embodiments.

Spinal cord stimulation can be accomplished utilizing either percutaneous leads and/or laminotomy type leads that comprise a paddle. Percutaneous leads commonly have two or more equally spaced electrodes which are placed above the dura layer through the use of a Touhy-like needle. For insertion, the Touhy-like needle is passed through the skin between desired vertebrae to open above the dura layer.

In contrast to the percutaneous leads, laminotomy leads have a paddle configuration and typically possess a plurality of electrodes (for example, two, four, eight, sixteen or thirty-two) arranged in one or more columns. Implanted laminotomy leads are commonly transversely centered over the physiological midline of a patient. In such position, multiple columns of electrodes are well suited to address both unilateral and bilateral pain, where electrical energy may be administered using either column independently (on either side of the midline) or administered using both columns to create an electric field which traverses the midline. A multi-column laminotomy lead enables reliable positioning of a plurality of electrodes, and in particular, a plurality of electrode columns that do not readily deviate from an initial implantation position.

Laminotomy leads require a surgical procedure for implantation. The surgical procedure, or partial laminectomy, requires the resection and removal of certain vertebral tissue to allow both access to the dura and proper positioning of a laminotomy lead. The laminotomy lead offers a more stable platform, which is further capable of being sutured in place that tends to migrate less in the operating environment of the human body. Depending on the position of insertion, however, access to the dura may only require a partial removal of the ligamentum flavum at the insertion site. In some embodiments, two or more laminotomy leads may be positioned within the epidural space, and the leads may assume any relative position to one another.

In certain embodiments, the stimulation leads may be placed subcutaneously on the patient's head. For example, one or more stimulation leads can be implanted subcutaneously such that one or more stimulation electrodes are positioned in communication with a dermatome area, for example (C1, C2, C3) or cervical nerve roots (e.g., C1, C2, C3) or cranial nerves (e.g., olfactory nerve, optic, nerve, oculomotor nerve, trochlear nerve, trigeminal nerve, abducent nerve, facial nerve, vestibulocochlear nerve, glossopharyngeal nerve, vagal nerve, accessory nerve, and hypoglossal nerve) and/or occipital area For example, one or more stimulation electrodes are positioned in the C2 dermatome area/C3 dermatome area, subcutaneously, but superior to the galea. Within certain areas of the C2 dermatome area or occipital or occiput area, there is little or no muscle, this area primarily consists of fat, fascia, perostium, and neurovascular structures (e.g., galea). More specifically, the electrode can be implanted in a subcutaneous fashion such that the electrode is positioned below the skin, above the bone on the back of the head or superior to the periosteum. On the back of the head, the probe is positioned in the C2 dermatome area or positioned at the back of the patient's head at about the level of the ear.

C. Brainstem Stimulation

Implantation of a stimulation lead in communication with the predetermined brainstem area can be accomplished via a variety of surgical techniques that are well known to those of skill in the art. For example, an electrical stimulation lead can be implanted on, in, or near the brainstem by accessing the brain tissue through a percutaneous route, an open craniotomy, or a burr hole. Where a burr hole is the means of accessing the brainstem, for example, stereotactic equipment suitable to aid in placement of an electrical stimulation lead on, in, or near the brainstem may be positioned around the head. Another alternative technique can include, a modified midline or retrosigmoid posterior fossa technique.

In certain embodiments, electrical stimulation lead is located at least partially within or below the dura mater adjacent the brainstem. Alternatively, a stimulation lead can be placed in communication with the predetermined brainstem area by threading the stimulation lead up the spinal cord column, as described above, which is incorporated herein.

Still further, a predetermined brainstem area can be indirectly stimulated by implanting a stimulation lead in communication with a cranial nerve (e.g., olfactory nerve, optic, nerve, oculomotor nerve, trochlear nerve, trigeminal nerve, abducent nerve, facial nerve, vestibulocochlear nerve, glossopharyngeal nerve, vagal nerve, accessory nerve, and the hypoglossal nerve) as well as high cervical nerves (cervical nerves have anastomoses with lower cranial nerves) such that stimulation of a cranial nerve indirectly stimulates the predetermined brainstem tissue. Such techniques are further described in U.S. Pat. Nos. 6,721,603; 6,622,047; and 5,335,657 each of which are incorporated herein by reference.

IV. Generation of Stimulation Parameters and Modifications Thereof

Conventional IMDs can be modified to apply 1/f^{(-β)} noise stimulation, or 1/f^{(-β)} noise stimulation in combination with individual peak frequencies (e.g., alpha, theta, delta, beta or gamma) or combination of 1/f^{(-β)} noise stimulation combined with burst or tonic stimulation to nerve tissue of a patient by modifying the software instructions and/or stimulation parameters stored in the devices. Specifically, conventional IMDs typically include a microprocessor and a pulse generation module. The pulse generation module generates the electrical pulses according to a defined pulse width and pulse amplitude and applies the electrical pulses to defined electrodes through switching circuitry and the wires of a stimulation lead. The microprocessor controls the operations of the pulse generation module according to software instructions stored in the device and accompanying stimulation parameters. Examples of commercially available IMDs that can be modified according to some embodiments include the Precision® marketed and sold by Boston Scientific. Other IMDs can include, Algovita® formerly marketed and sold by Nuvectra, Nalu® Neurostimulation System, or Medtronic's Activa® marketed and sold by Medtronic.

These IMDs can be adapted by modifying the software instructions provided within the IMDs used to control the operations of the devices. In some embodiments, software is provided within the IMD to retrieve or generate a stream of digital values that define a waveform according to the desired power spectral density. This stream of values is then employed to control the amplitude of successive stimulation pulses generated by the IMD. The software may include a pseudo-random number generator according to known algorithms to generate the stream of digital values. Alternatively, one or more streams of digital values having the desired power spectral density may be generated offline and stored in memory of the IMD (in a compressed or other suitable format). The software of the IMD may retrieve the values from memory for control of the amplitude of the output pulses of the IMD. Alternatively, an external conventional IMD can be used (for example, the DS8000™ digital stimulator available from World Precision Instruments) to generate the desired electrical stimulation. For example, a custom waveform may be generated offline on a personal computer and imported into the digital stimulator for pulse generation. Signal parameters may be inputted, such as 1/f^{(-β)} noise spectrum, for example FIG. 1A or FIG. 1B into suitable waveform generating software to generate the stream of digital values. Alternatively, depending upon the capabilities of the external digital stimulator, the stream of digital values may be calculated on board the processor of the external digital stimulator.

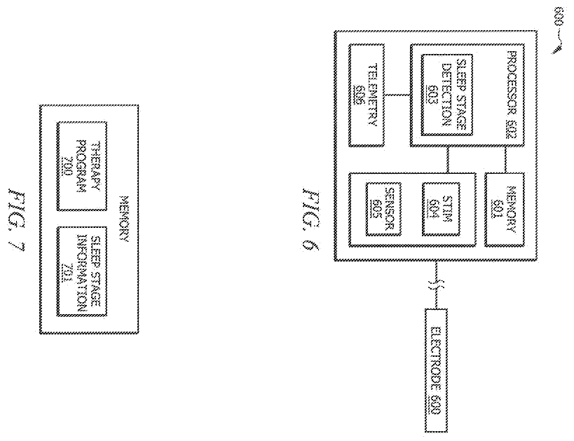

FIG. 4 depicts an exemplary IMD that can be used to provide the desired stimulation. Signal parameters are inputted, such as 1/f^{(-β)} noise spectrum, for example FIG. 1A or FIG. 1B, into the software or memory 410 and the desired wave pattern or signals are generated using microprocessor 420. A standard digital-to-analog converter 430 receives the calculated digital signals and generates analog output pulses corresponding to the values of the digital signals. The generated output pulses may be outputted from the IMD through an output capacitor. Optionally, any suitable filter 440 can be used to smooth or shape the signals; however, unsmoothed or unfiltered signals can be transmitted to the switching circuitry 450 which provides the signals to the electrodes 300 thereby stimulating the neuronal tissue using the desired 1/f^{(-β)} noise pattern.

Another example of the IMD stimulator design is disclosed in U.S. Pat. No. 8,996,117, which discloses a waveform generator with scalable waveform features. The IMD disclosed in U.S. Pat. No. 8,996,117 is well suited to deliver the 1/f^{(-β)} noise waveforms. Those skilled in the art recognize that other IMDs such as the IMD disclosed in U.S. Pat. No. 4,793,353 and including those mentioned within this disclosure and those on the market that are not specifically mentioned herein are able to deliver the 1/f^{(-β)} noise waveform as well. U.S. Pat. Nos. 8,996,117 and 4,793,353 are incorporated by reference herein in full. As those skilled in the art will appreciate, the example IMDs in U.S. Pat. Nos. 8,996,117 and 4,793,353, would provide a representation of 1/f^{(-β)} noise as disclosed herein.

In addition to providing a stimulation waveform similar to that of 1/f^{(-β)} noise spectrum; it may be desirable to modify the 1/f^{(-β)} noise waveform stimulation pattern. Such modifications can utilize the addition of peak frequencies, such as the addition of an alpha, beta, theta, and/or delta peaks to 1/f^{(-β)} noise spectrum waveform, see for example, FIGS. XXA and XXB. Such frequency peaks can be obtained by using standard peaks or individualizing the frequency peaks. Such information can be communicated to the microprocessor 420 via the software component 410. Thus, the data communicated can comprise standard frequency peaks or comprise individualized frequency peaks or patient specific. The patient specific frequency peaks can be obtained off-line or in real time or on-line, for example prior to implantation or at any time point after implantation, for example, during the initial programming of the IMD. Any suitable signal processing technique may be employed to add the appropriate spectral peaks. For example, a suitable filter may be applied to the noise signal. Alternatively, a separate signal may be generated with a spectral peak about the desired frequency and the separate signal may be added to or superimposed on the noise signal.

With reference to FIG. 5, with electrodes disposed near, adjacent to directly next to or within the target neuronal tissue, for example, brain tissue, some representative embodiments utilize the detection and analysis of neuronal activity, such as EEG measurements. Specifically, terminals of the lead, such as an EEG lead, may be coupled to electrodes 501 using respective conductors 500 to external controller that contains suitable circuitry to analyze neuronal activity, for example, an EEG analyzer can be included in the external controller in which the analyzer functions are adapted to receive EEG signals from the electrodes and process the EEG signals to identify frequency peaks, such as LORETA software can be used. Further signal processing may occur on a suitable computer platform within the external controller using available signal processing. The computer platform may include suitable signal processing algorithms (e.g., time domain segmentation, FFT processing, windowing, logarithmic transforms, etc.). Further platforms or algorithms to modify the signals are included in the modification algorithms (e.g., envelope modification, etc). User interface software may be used to present the processed neuronal activity (i.e., specific peak frequency) and combine a specific peak frequency with the 1/f^{(-β)} noise waveform patterns to the transmitter 503 which then transmits, for example, via radio frequency to the IMD 604 which is adapted to provide the 1/f^{(-β)} noise waveform patterns with the peak frequency to achieve stimulation of the target neuronal tissue via electrode 400. This procedure can be performed on-line or off-line. Additionally, IMD 504 preferably comprises circuitry such as an analog-to-digital (AD) converter, switching circuitry, amplification circuitry, transmitters, and/or filtering circuitry.

Still further, it may be desirable to utilize an implantable device that is capable of performing only the functions of the external controller or that is capable of performing the functions of the external controller and the functions of an IMD, all in one. When the implantable device is only performing only those functions of the external controller, those of skill in the art can modify an implantable device such that it is capable of detecting/sampling and processing of the signal's representative of the neuronal activity/EEG activity. Such a device may include a microprocessor that is capable of performing these activities as well as a transmitter such that the signals can be transmitted via radiofrequency to another implantable device, such as described above in FIG. 5 that is capable of generating the desired signal to the target tissue. Thus, an EEG lead is placed or positioned near the target brain tissue via methods known to those of skill in the art. The EEG lead detects neuronal activity which is relayed to the processor that possesses sufficient computational capacity to collect the information obtained from the EEG electrode, process it to obtain the respective frequency peak desired and/or modulate the frequency peaks and transmit the frequency to an RF transmitter that transmits the respective information to microprocessor located in the stimulation IMD. Those skilled in the art will realize that for the combined IMD, the information will be processed by a microcontroller within the IMD and transferred to the correct processing circuitry to obtain the respective frequency peak desired and/or modulate the frequency peaks and stimulate according to the invention as described herein.

Another means to modify the 1/f^{(-β)} noise waveform pattern is to combine it with any variety of waveforms, such as sinewaves, arbitrary waveforms, tonic waveforms, or burst waveforms. Thus, an IMD can be implemented to apply either a variety of waveforms using a digital signal processor and one or several digital-to-analog converters. The waveforms could be defined in memory and applied to the digital-to-analog converter(s) for application through electrodes of the medical lead. The digital signal processor could scale the various portions of the waveform in amplitude and within the time domain (e.g., for the various intervals) according to the various waveform parameters. A doctor, the patient, or another user of waveform source may directly or indirectly input waveform parameters to specify or modify the nature of the waveforms provided. An example system able to perform this function is the former Nuvectra's Algovita system.

Thus, a microprocessor and suitable software instructions to implement the appropriate system control can be used to control waveform therapy in combination with 1/f^{(-β)} noise. The processor can be programmed to use “multiple programs sets” which are known in the art. A “program set” refers to a set of waveform parameters which define a waveform's pulse to be generated. For example, a program set defines the waveform's pulse amplitude, a pulse width, a pulse delay, pulse shape, frequency, and an electrode combination among other such parameters. The pulse amplitude refers to the amplitude for a given pulse and the pulse width refers to the duration of the pulse. The pulse delay represents an amount of delay to occur after the generation of the pulse (equivalently, an amount of delay could be defined to occur before the generation of a pulse). The waveform shape represents the design or shape of the waveform. The amount of delay represents an amount of time when no pulse generation occurs. The electrode combination defines the polarities for each output which, thereby, controls how a pulse is applied via electrodes of a lead. Other pulse parameters could be defined for each program set such as pulse type, repetition parameters, etc. Still further, the 1/f^{(-β)} noise waveform pattern alone or in combination with other waveforms may be implemented such that the stimulation occurs either sequentially, randomly or pseudo-sequentially over multiple poles or electrodes on the lead.

In certain embodiments, the waveform parameters may comprise a type of stimulation having a frequency in the range of about 0.01 Hz to about 1000 Hz in combination with a different type of stimulation having also a frequency in the range of about 0.01 Hz to about 1000 Hz but with a different design Those of skill in the art realize that the frequencies can be altered depending upon the capabilities of the IMDs that are utilized. More particularly, for example, burst stimulation may be at about 0.01 to 1000 Hz consisting of 2 to 10 spikes with 1 ms to 10 ms pulse width, 0.1 to 100 ms interspike interval in combination with 1/f^{(-β)} noise signals interspersed between or around the burst or prior to or after the burst or in any variation thereof depending upon the efficacy of treatment. Still further, 1/f^{(-β)} noise signals or stimulation paradigm as described herein may be used in combination with about 0.01 to 1000 Hz tonic stimulation interspersed between or around the 1/f^{(-β)} noise signals or stimulation paradigm, or any variation thereof depending upon the efficacy of treatment and the capabilities of the IMD.

Still further, those of skill in the art recognize that burst firing refers to an action potential that is a burst of high frequency spikes (50-1000 Hz) (Beurrier et al., 1999). Burst firing acts in a non-linear fashion with a summation effect of each spike and tonic firing refers to an action potential that occurs in a linear fashion.

Yet further, a particular stimulation paradigm can refer to a period in the stimulation train that has a much higher discharge rate than surrounding periods in the spike train (N. Urbain et al., 2002). An example of a particular paradigm, burst can refer to a plurality of groups of spike pulses. A burst is a train of action potentials that, possibly, occurs during a ‘plateau’ or ‘active phase’, followed by a period of relative quiescence called the ‘silent phase’ (Nunemaker, Cellscience Reviews Vol 2 No. 1, 2005.) Thus, a burst paradigm may comprise of spikes having an inter-spike interval in which the spikes are separated by 0.1 milliseconds to about 100 milliseconds. Those of skill in the art realize that the inter-spike interval can be longer or shorter. Yet further, those of skill in the art also realize that the spike rate within the burst does not necessarily occur at a fixed rate; this rate can be variable. A spike refers to an action potential. Yet further, a “burst spike” refers to a spike that is preceded or followed by another spike within a short time interval (Matveev, 2000), in other words, there is an inter-spike interval, in which this interval is generally about 100 ms but can be shorter or longer, for example 0.1 milliseconds.

Still further, it may be of interest to use a system that includes a processor that determines whether the patient is in a sleep state, and controls therapy based upon the sleep state. The sleep state may be relevant for 1/f^{(-β)} noise therapy if during a given sleep stage the patient's frequency spectrum changes, for example, the high frequency component in gamma is decreased. This suggests that there are less prediction errors and therefore in this embodiment IMD may automatically adjust the value of β for 1/f^{(-β)} noise from a value of 1 to a value of 2 or even steeper stimulation.

As referred to herein, the sleep state may refer to a state in which patient is intending on sleeping (e.g., initiating thoughts of sleep), is at rest, is attempting to sleep or has initiated sleep and is currently sleeping. In addition, the processor may determine a sleep stage of the sleep state based on a biosignal detected within brain the patient and control therapy delivery to patient based on a determined sleep stage. Examples of biosignals include, but are not limited to, electrical signals generated from local field potentials within one or more regions of brain, such as, but not limited to, an electroencephalogram (EEG) signal or an electrocorticogram (ECOG) signal. The biosignals that are detected may be detected within the same tissue site of brain as the target tissue site for delivery of electrical stimulation. In other examples, the biosignals may be detected within another tissue site.

Within a sleep state, the patient may be within one of a plurality of sleep stages. Example sleep stages include, for example, Stage 1 (also referred to as Stage N1 or S1), Stage 2 (also referred to as Stage N2 or S2), Deep Sleep (also referred to as slow wave sleep), and rapid eye movement (REM). The Deep Sleep stage may include multiple sleep stages, such as Stage N3 (also referred to as Stage S3) and Stage N4 (also referred to as Stage S4). In some cases, the patient may cycle through the Stage 1, Stage 2, Deep Sleep, REM sleep stages more than once during a sleep state. The Stage 1, Stage 2, and Deep Sleep stages may be considered non-REM (NREM) sleep stages.

FIG. 8 shows an exemplary implantable IMD 800 that can be used to determine a stage of sleep and adjust therapy. For example, the device may include, processor 802, memory 801, stimulation generator 804, sensing module 805, telemetry module 806, and sleep stage detection module 803. Although sleep stage detection module 803 is shown to be a part of processor 802 in FIG. 7, in other examples, sleep stage detection module 803 and processor 802 may be separate components and may be electrically coupled, e.g., via a wired or wireless connection.

Memory 801, as shown in FIG. 9, may include any volatile or non-volatile media, such as a random access memory (RAM), read only memory (ROM), non-volatile RAM (NVRAM), electrically erasable programmable ROM (EEPROM), flash memory, and the like. Memory 801 may store instructions for execution by processor 802 and information defining therapy delivery for the patient, such as, but not limited to, therapy programs or therapy program groups, information associating therapy programs with one or more sleep stages, thresholds or other information used to detect sleep stages based on biosignals, and any other information regarding therapy of the patient. Therapy information may be recorded in memory 801 for long-term storage and retrieval by a user. As described in further detail with reference to FIG. 9, memory 801 may include separate memories for storing information, such as separate memories for therapy programs 900, and sleep stage information 901. Yet further, other memories that may be stored may include patient information, such as information relating to specific peak frequencies, or information relating to 1/f^{(-β)} noise.

It is also envisaged that the recording electrode can be used to record or detect pathological beta and gamma activity, in an isolated way or in a cross-frequency coupled way, or to detect a sleep stage or when a subject is not in a sleep stage, the recording electrode can be used to detect a change in the normal spectral composition of the noise and adjust the parameters of the stimulation therapy, for example, adjust the stimulation factors such as drowsiness, stress, depression, excitement, arousal, alcohol or other drug intake etc.

V Treating Neurological Conditions

The present stimulation method acts to stimulate neuronal tissue which in turn stimulate the neuronal tissue to cause/allow the tissue to act in the best interest of the host through use of the its natural mechanisms.

Accordingly, the present methods and/or devices relate to modulation of neuronal activity to affect neurological, neuropsychological or neuropsychiatric activity. The present method finds particular application in the modulation of neuronal function or processing to affect a functional outcome. The modulation of neuronal function is particularly useful with regard to the prevention, treatment, or amelioration of neurological, psychiatric, psychological, conscious state, behavioral, mood, and thought activity (unless otherwise indicated these will be collectively referred to herein as “neurological activity” which includes “psychological activity” or “psychiatric activity”). When referring to a pathological or undesirable condition associated with the activity, reference may be made to a neurological disorder which includes “psychiatric disorder” or “psychological disorder” instead of neurological activity or psychiatric or psychological activity. Although the activity to be modulated usually manifests itself in the form of a disorder such as a attention or cognitive disorders (e.g., Autistic Spectrum Disorders); mood disorder (e.g., major depressive disorder, bipolar disorder, and dysthymic disorder) or an anxiety disorder (e.g., panic disorder, posttraumatic stress disorder, obsessive-compulsive disorder and phobic disorder); neurodegenerative diseases (e.g., multiple sclerosis, Alzheimer's disease, amyotrophic lateral sclerosis (ALS), Parkinson's disease, Huntington's Disease, Guillain-Barre syndrome, myasthenia gravis, and chronic idiopathic demyelinating disease (CID)), movement disorders (e.g., dyskinesia, tremor, dystonia, chorea and ballism, tic syndromes, Tourette's Syndrome, myoclonus, drug-induced movement disorders, Wilson's Disease, Paroxysmal Dyskinesias, Stiff Man Syndrome and Akinetic-Ridgid Syndromes and Parkinsonism), epilepsy, tinnitus, pain, phantom pain, diabetes neuropathy, one skilled in the art appreciates that the invention may also find application in conjunction with enhancing or diminishing any neurological or psychiatric function, not just an abnormality or disorder. Neurological activity that may be modulated can include, but not be limited to, normal functions such as alertness, conscious state, drive, fear, anger, aggression, anxiety, repetitive behavior, impulses, urges, obsessions, euphoria, sadness, and the fight or flight response, as well as instability, vertigo, dizziness, fatigue, photophobia, concentration dysfunction, memory disorders, including dementias such as Alzheimer or Lewy body dsease, headaches and facial pains, dizziness, irritability, fatigue, visual disturbances, sensitivity to noise (misophonia, hyperacusis, photophobia), judgment problems, depression, symptoms of traumatic brain injury (whether physical, emotional, social or chemical), autonomic functions, which includes sympathetic and/or parasympathetic functions (e.g., control of heart rate), somatic functions, and/or enteric functions. Thus, the present methods and/or devices encompass modulation of central and/or peripheral spinal and autonomic nervous systems. In another embodiment the stimulation may be used to treat personality disorders, including Cluster A: paranoid, Schizoid and Schizotypal, Cluster B (dramatic): Antisocial, Borderline, Histrionic, Narcissistic, and Cluster C: (anxious) Avoidant, Dependent, Obsessive-compulsive or Not specified: Depressive, Haltlose, Passive-aggressive, Sadistic, Self-defeating and Psychopathic personality disorders.

Other neurological disorders can include, but are not limited to headaches, for example, migraine, trigeminal autonomic cephalgia (cluster headache (episodic and chronic)), paroxysmal hemicrania (episodic and chronic), hemicrania continua, SUNCT (short-lasting unilateral neuralgiform headache with conjunctiva! injection and tearing), cluster tic syndrome, trigeminal neuralgia, tension type headache, idiopathic stabbing headache, etc. The neurostimulation device can be implanted intracranially or peripherally, for example, but not limited to implanting a neurostimulation device occipitally or frontally for the treatment of headaches and facial pain.

Autonomic and/or enteric nervous system disorders that can be treated using the stimulation system and/or method of the present invention include, but are not limited to hypertension, neurosis cordis or heart rhythm disorders, obesity, gastrointestinal motion disorders, respiratory disorders, diabetes, sleep disorders, snoring, incontinence both urologic and gastrointestinal, sexual dysfunction, chronic fatigue syndrome, fibromyalgia, whiplash associated symptoms, post-concussion syndrome, posttraumatic stress disorder etc.

Yet further immunological disorders may also be treated using the stimulation system and/or method of the present invention. This is based on the fact that the immune system senses antigens coordinates' metabolic, endocrine and behavioral changes that support the immune system and modulates the immune system via neuroendocrine regulation and direct immune cell regulation. Such immunological disorders include, such as allergy, rhinitis, asthma, rheumatoid arthritis, psoriasis arthritis, lupus erythematosus disseminatus, multiple sclerosis and other demyelinating disorders, autoimmune thyroiditis, Crohn's disease, diabetes mellitus etc. in this embodiment the stimulation may be at the autonomic nerves, the vagus nerve, C2 or C2 nerve, the spinal cord or inside the brain where the central control is regulated, for example the posterior cingulate cortex, anterior cingulate cortex, insula, hypothalamus.

Still further tumoral disorders, both malignant and benign may also be treated using the stimulation system and/or method of the present invention. This is based on the fact that tumoral behavior is linked to immunological function. This is seen in immunodeficiency syndromes such as AIDS and hematological disorders, where multiple and different tumors develop. In this setting neuromodulation could indirectly influence tumoral behavior.

Yet further neuroendocrine disorders may also be treated using the stimulation system and/or method of the present invention. Such disorders are stress reactions, hypothalamic-pituitary axis dysfunction, etc.

Yet further functional disorders may also be treated using the stimulation system and/or method of the present invention. Such disorders can be anorexia, bulimia, phobias, addictions, paraphilia, psychosis, depression, bipolar disorder, kleptomania, aggression, or antisocial sexual behavior. One skilled in the art appreciates that the invention may also find application in conjunction with enhancing or diminishing any neurological or psychiatric function, not just an abnormality or disorder.

Using the above described stimulation system, the predetermined site or target area is stimulated in an effective amount or effective treatment regimen to decrease, reduce, modulate or abrogate the neurological disorder or condition. Thus, a subject or patient is administered a therapeutically effective stimulation so that the subject has an improvement in the parameters relating to the neurological disorder or condition including subjective measures such as, for example, neurological examinations and neuropsychological tests (e.g., Minnesota Multiphasic Personality Inventory, Beck Depression Inventory, Mini-Mental Status Examination (MMSE), Hamilton Rating Scale for Depression, Wisconsin Card Sorting Test (WCST), Tower of London, Stroop task, MADRAS, CGI, N-BAC, or Yale-1/f^{(-β)} noise Obsessive Compulsive score (Y-BOCS)), motor examination, visual analog scale (VAS) and cranial nerve examination, and objective measures including use of additional psychiatric medications, such as anti-depressants, or other alterations in cerebral blood flow or metabolism and/or neurochemistry.

Patient outcomes may also be tested by health-related quality of life (HRQL) measures: Patient outcome measures that extend beyond traditional measures of mortality and morbidity, to include such dimensions as physiology, function, social activity, cognition, emotion, sleep and rest, energy and vitality, health perception, normal eating habits or behaviors (i.e., regained appetite or reduced appetite) and general life satisfaction. (Some of these are also known as health status, functional status, or quality of life measures).

Treatment regimens may vary as well, and often depend on the health and age of the patient. Obviously, certain types of disease will require more aggressive treatment, while at the same time; certain patients cannot tolerate more taxing regimens. The clinician will be best suited to make such decisions based on the known subject's history.

For purposes of this invention, beneficial or desired clinical results include, but are not limited to, alleviation of symptoms, improvement of symptoms, diminishment of extent of disease, stabilized (i.e., not worsening) state of disease, delay or slowing of disease progression, amelioration or palliation of the disease state, and remission (whether partial or total), whether objective or subjective. The improvement is any observable or measurable improvement, or any subjective improvement reported by the implanted person. Thus, one of skill in the art realizes that a treatment may improve the patient condition but may not be a complete cure of the disease.

In certain embodiments, in connection with improvement in one or more of the above or other neurological disorders, the electrical stimulation may have a “brightening” effect on the person such that the person looks better, feels better, moves better, thinks better, and otherwise experiences an overall improvement in quality of life, (e.g., self-confidence, alleviating shyness, distrust etc). In another embodiment this stimulation may be used as enhancement of normal functions if the person desires so.

In certain embodiments, the neuromodulation method described herein is utilized to treat a subject suffering or suspected of suffering in general, or more specifically from tinnitus. Thus, a subject is administered a therapeutically effective stimulation so that the subject has an improvement in the parameters relating to tinnitus including informal questioning of the subject, formal subjective testing and analysis according to one or more audiology test, for example the Goebel tinnitus questionnaire or other validated tinnitus questionnaires, audiometry, tinnitus matching, impedance, BAEP, and OAE. The improvement is any observable or measurable improvement. Thus, one of skill in the art realizes that a treatment may improve the patient condition but may not be a complete cure of the disease.

In other embodiments, the neuromodulation method described herein is utilized to treat a subject suffering from or suspected of suffering from pain (chronic or otherwise). One example of a method for pain measurement is the use of the Visual Analog Scale (VAS). In the VAS patients are asked to rank their pain by making a mark on a bar that is labeled “no pain” on one end, and “pain as bad as possible” on the other end. Patients may mark the bar anywhere between the two opposite poles of perceived pain sensation. This mark can then be given any quantitative value such as fractional, decimal or integer values by the clinician and used as a semi-quantitative pain measurement. In various tests for pain severity, patients may rank their pain on a scale between zero and ten, by a scale of faces depicting various emotions from happy to very sad and upset, and by answering a variety of questions describing the pain. In preferred embodiments, the patient's pain is assessed prior to and during a trial implantation procedure. In other embodiments, informal subjective questioning of the person, and/or formal subjective testing and analysis may be performed to determine whether the subject's pain has sufficiently improved throughout trial stimulation.

In addition to utilizing pain scores and grading and objective measures including use of additional pain medications (e.g., reduction in the amount of medication consume or elimination of the consumption of pain medications), other methods to determine improvement of a patient's pain may comprise administering various standardized questionnaires or tests to determine the patient's neuropsychological state as described above.

If the subject's neurological disorder/disease has not sufficiently improved, or if the reduction of the neurological disorder/disease is determined to be incomplete or inadequate during an intra-implantation trial stimulation procedure, stimulation lead may be moved incrementally or even re-implanted, one or more stimulation parameters may be adjusted, or both of these modifications may be made and repeated until at least one symptom associated with the neurological disorder/disease has improved.

Where appropriate, post-implantation trial stimulation may be conducted to determine the efficacy of various types of burst and tonic stimulation. Examples of efficacy metrics may include the minimum required voltage for a given protocol to achieve maximum and/or therapeutic benefits to the neurological disease and/or disorder. Efficacy metrics may also include a measurement of the presence and/or degree of habituation to a given protocol over one or more weeks or months, and any necessary modifications made accordingly. Such assessments can be conducted by any suitable programming device. One such as suitable device is that described in U.S. Pat. No. 9,144,680, which is incorporated by reference here in full. Utilizing such a program allows an optimal stimulation therapy to be obtained at minimal power. This ensures a longer battery life for the implanted systems.

In certain embodiments, it may be desirable for the patient to control the therapy to optimize the operating parameters to achieve increased or optimized the treatment. For example, the patient can alter the pulse frequency, pulse amplitude and pulse width using a handheld radio frequency or Bluetooth or wifi enabled device that communicates with the IMD. Once the operating parameters have been altered by the patient, the parameters can be stored in a memory device to be retrieved by either the patient or the clinician. Yet further, particular parameter settings and changes therein may be correlated with particular times and days to form a patient therapy profile that can be stored in a memory device. In another embodiment remote programming via the cloud may be performed by the treating physician or health care provider.

As will be recognized by those skilled in the art, the innovative concepts described in the present application can be modified and varied over a wide range of applications. Accordingly, the scope of patented subject matter should not be limited to any of the specific exemplary teachings discussed above but is instead defined by the following claims.

What is claimed is:

1. A method of stimulating nerve tissue of a patient using an IMD, the method comprising:
generating, by an IMD containing a pulse generator, a stimulus that comprises a signal that is produced from a frequency spectrum having a power spectral density per unit of bandwidth proportional to 1/f^{(-β)}, wherein β is any real number but excludes 0;
providing the stimulus from the IMD containing a pulse generator to at least one stimulation lead; and
applying the stimulus to nerve tissue of the patient via one or several electrodes of at least one stimulation lead.

2. The method of claim 1, wherein β=1

3. The method of claim 1, wherein β=2

4. The method of claim 1, wherein β>2

5. The method of claim 1, wherein the stimulus is combined with at least one pulse stimulus to be repeated in a tonic manner.

6. The method of claim 1, wherein the stimulus is combined with a burst stimulus that comprises a plurality of groups of spike pulses, each charge balanced or charge balanced at the end of the group of monophasic spikes.

7. The method of claim 1, wherein the stimulus is combined with at least one pulse stimulus to be repeated in a tonic manner and with a burst stimulation that comprises a plurality of groups of spike pulses, each charge balanced or charge balanced at the end of the group of monophasic spikes.

8. The method of claim 1, wherein the stimulus is modulated at any specific frequency, either by selective power increase, envelope modulation or adding more tonic or burst stimuli of this frequency.

9. A method of stimulating nerve tissue of a patient using an IMD containing a pulse generator, the method comprising:
storing, in the IMD containing a pulse generator, one first stimulation parameter that defines a frequency to be used as the lower bound of a frequency spectrum;
storing, in the IMD containing a pulse generator, one second stimulation parameter that defines a frequency to be used as the upper bound of a frequency spectrum;
generating, by IMD containing a pulse generator, a stimulus that comprises a frequency spectrum between the first stimulation parameter and second stimulation parameter, wherein the frequency and power of the frequency spectrum are proportional;
providing the stimulus from the implantable IMD containing a pulse generator to at least one stimulation lead; and
applying the stimulus to nerve tissue of the patient via at least one electrode of at least one stimulation lead wherein the stimulus is further defined as having a power spectral density per unit of bandwidth is proportional to 1/f^{(-β)}, wherein β is any real number but excludes 0.

10. The method of claim 9, wherein β=1.

11. The method of claim 9, wherein β=2.

12. The method of claim 9, wherein β>2.

13. The method of claim 9, wherein the stimulus is combined with at least one stimulation pulse to be repeated in a tonic manner.

14. The method of claim 9, wherein the stimulus is combined with a burst stimulus that comprises a plurality of groups of spike pulses.

15. A method of stimulating nerve tissue of a patient using an IMD containing a pulse generator, the method comprising:
storing, in the IMD containing a pulse generator, one first stimulation parameter that defines a frequency to be used as the lower bound of a frequency spectrum;
storing, in the IMD containing a pulse generator, one second stimulation parameter that defines a frequency to be used as the upper bound of a frequency spectrum;
storing, in IMD containing a pulse generator, one third stimulation parameter that defines a frequency at which a peak of a pre-determined amplitude is to occur;
generating, by IMD containing a pulse generator, a stimulus that comprises a frequency spectrum between the first stimulation parameter and second stimulation parameter, wherein the power spectral density per unit of bandwidth is proportional to 1/f^{(-β)}, wherein β is any real number but excludes 0, and wherein a peak of a predetermined amplitude occurs at the frequency defined by the third stimulation parameter;
providing the stimulus from the IMD containing a pulse generator to at least one stimulation lead; and
applying the stimulus to nerve tissue of the patient via one or several electrodes of at least one stimulation lead.

16. The method of claim 16, wherein the noise stimulus is combined with at least one stimulation pulse to be repeated in a tonic manner and with a burst stimulation that comprises a plurality of groups of spike pulses.

17. The method of claim 16, wherein the peak occurs at a frequency between 0 and 4 Hertz.

18. The method of claim 16, wherein the peak occurs at a frequency between 4 and 7 Hertz.

19. The method of claim 16, wherein the peak occurs at a frequency between 8 and 12 Hertz.

20. The method of claim 16, wherein the peak occurs at a frequency between 12 and 30 Hertz.
